(19) United States
(12) Patent Application Publication
(10) Pub. No.: US 2024/0335168 A1
(43) Pub. Date: Oct. 10, 2024

(54) SYSTEM AND METHODS FOR PERSONALIZED NON-ENZYME SIGNAL COMPENSATION

(71) Applicant: Dexcom, Inc., San Diego, CA (US)

(72) Inventors: Qi AN, San Diego, CA (US); Kevin CHENG, San Diego, CA (US); Devon M. HEADEN, San Diego, CA (US); Anita Hanzong SHIEH, San Diego, CA (US)

(21) Appl. No.: 18/627,759

(22) Filed: Apr. 5, 2024

Related U.S. Application Data
(60) Provisional application No. 63/494,976, filed on Apr. 7, 2023.

(51) Int. Cl. A61B 5/00; A61B 5/145; A61B 5/1473

(52) U.S. Cl. CPC A61B 5/7203; A61B 5/14532; A61B 5/14546; A61B 5/14735; A61B 5/6801; A61B 5/7405; A61B 5/742; A61B 5/7455; A61B 2560/045; A61B 2560/0468

(57) ABSTRACT

The present disclosure describes a continuous analyte monitoring system that may monitor, generate, and analyze data for analytes (e.g., glucose and/or lactate) and non-enzymes simultaneously. In certain aspects, an analyte sensor system includes a first electrode, a second electrode, and a sensor electronics module. The first electrode generates a first analyte signal stream. The second electrode generates a non-enzyme signal stream indicating a level of a non-enzyme over time. The sensor electronics module determines a level of a first analyte based on the first analyte signal stream and adjusts the level of the first analyte based on the non-enzyme signal stream.

### CROSS-REFERENCE TO RELATED APPLICATIONS

This application claims priority to U.S. Provisional Patent Application No. 63/494,976, filed Apr. 7, 2023, which is hereby assigned to the assignee hereof and hereby expressly incorporated by reference herein in its entirety as if fully set forth below and for all applicable purposes.

### BACKGROUND

Diabetes mellitus is a metabolic condition relating to the production or use of insulin by the body. Insulin is a hormone that allows the body to use glucose for energy, or store glucose as fat.

When a person eats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis—a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using monitoring systems that measure glucose levels, which inform the identification and/or prediction of adverse glycemic events, such as hyperglycemia and hypoglycemia, and the type of guidance provided to the patient.

For example, such monitoring systems may utilize a continuous glucose monitor (CGM) to measure a patient's glucose levels over time. The measured glucose levels may then be processed by the monitoring system to identify and/or predict adverse glycemic events, and/or to provide guidance to the patient for treatment and or actions to abate or prevent the occurrence of such adverse glycemic events. For example, trends, statistics, or other metrics may be derived from the glucose levels and used to identify and/or predict adverse glycemic events. Or, in certain cases, the glucose levels themselves may be used to identify and/or predict adverse glycemic events.

Even with the systems described above, however, the management of diabetes presents many challenges for patients, clinicians, and caregivers, as a confluence of various factors can impact a patient's glucose levels, thus affecting the accuracy of glycemic event prediction and the guidance provided by diagnostics systems.

### SUMMARY

The present disclosure describes a continuous analyte monitoring system that may monitor, generate, and analyze data for analytes (e.g., glucose and/or lactate) and non-enzymes simultaneously. According to an embodiment, an analyte sensor system includes a first electrode, a second electrode, and a sensor electronics module. The first electrode generates a first analyte signal stream. The second electrode generates a non-enzyme signal stream indicating a level of a non-enzyme over time. The sensor electronics module determines a level of a first analyte based on the first analyte signal stream and adjusts the level of the first analyte based on the non-enzyme signal stream.

According to another embodiment, an analyte sensor system includes a first electrode, a second electrode, and a sensor electronics module. The first electrode is covered by a membrane and generates a first analyte signal stream. The second electrode generates a non-enzyme signal stream indicating a level of non-enzymes over time. The sensor electronics module determines a level of a first analyte based on the first analyte signal stream and adjusts the level of the first analyte based on the non-enzyme signal stream.

According to another embodiment, a method includes generating a first analyte signal stream using a first electrode and generating a non-enzyme signal stream using a second electrode. The non-enzyme signal stream indicates a level of non-enzymes over time. The method also includes determining, by a sensor electronics module, a level of a first analyte based on the first analyte signal stream and adjusting, by the sensor electronics module, the level of the first analyte based on the non-enzyme signal stream.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a diagram conceptually illustrating an example continuous analyte monitoring system including example continuous analyte sensors with sensor electronics, in accordance with certain aspects of the present disclosure.

FIG. 2 is a block diagram that illustrates electronics associated with the sensor system of FIGS. 1 and 2, in accordance with certain aspects of the present disclosure.

FIG. 3 schematically illustrates an example configuration of a continuous analyte monitoring system, according to some embodiments disclosed herein.

FIG. 4 illustrates a flow diagram depicting an example method for adjusting a determined level of an analyte, according to some embodiments disclosed herein.

FIG. 5 illustrates a flow diagram depicting an example method for adjusting a determined level of an analyte, according to some embodiments disclosed herein.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In a continuous glucose monitor, a transcutaneous continuous glucose sensor that is inserted into the interstitial fluid is used to monitor a patient's glucose levels, thereby, providing glucose measurements reflective of the physiological state of the patient. Monitoring multiple analytes, in addition to glucose, may provide a more complete picture of the physiological state of the patient. An analyte may be understood as any substance of interest that is to be measured or is being measured. Examples of such analytes include, ketones, lactate, insulin, electrolytes, creatinine, as well as a number of other biomarkers including proteins, metabolites, and nucleic acids. The sensor electrodes of the transcutaneous continuous glucose sensor may interact with the desired analyte(s), e.g., through aptamers (single-stranded DNA or RNA molecules that bind to a specific analyte). An analyte of particular interest may be referred to as a target analyte. For example, in a transcutaneous continuous glucose sensor, the target analyte may be glucose.

Performance of a transcutaneous continuous glucose sensor, however, may be affected by other analytes (e.g., other substances) present in the body, such as electrochemical substances (e.g., acetaminophen) present in the interstitial fluid of the patient. These other analytes may also be referred to as “non-target enzymes” or “non-enzymes.” As discussed, sensor electrodes of a transcutaneous continuous glucose sensor may be inserted into the interstitial fluid to detect a desired analyte, and therefore, the electrodes are also exposed to these other substances in the interstitial fluid. The non-enzymes may interact with the sensor electrodes, creating signal noise. The signal noise may artificially increase or decrease the signal generated by the desired analyte, causing inaccurate sensor readings. These inaccurate sensor readings may then indicate false hypoglycemic or hyperglycemic events, interfere with analyte trend or pattern analysis, or lead to inaccurate insulin dosage calculations and delivery. Similar to a transcutaneous continuous glucose sensor, other transcutaneous continuous analyte sensors may similarly suffer from the same deficiencies.

Accordingly, the present disclosure provides a technical solution to these problems by providing a continuous analyte monitoring system that may monitor, generate, and analyze data for analytes (e.g., glucose and/or lactate) and non-enzymes simultaneously. The continuous analyte monitoring system includes at least two analyte sensors. A first analyte sensor monitors a first analyte (e.g., glucose) and generates a signal stream indicative of measurements of the first analyte (e.g., a concentration of the first analyte), while a second analyte sensor monitors a non-enzyme and generates a signal stream indicative of one or more measurements of the non-enzyme (e.g., a concentration of the non-enzyme). The continuous analyte monitoring system may determine a measurement of the first analyte based on the first analyte signal stream (e.g., based on a signal value of the first analyte signal stream), and then adjust the measurement for the first analyte based on the signal stream for the non-enzyme, automatically and without user intervention. This adjustment may include normalizing or subtracting a portion of the non-enzyme signal stream from the first analyte signal stream.

Additionally, in some embodiments, a third sensor may be used to generate a signal stream indicative of one or more measurements of a second analyte (e.g., a concentration of the second analyte), such as lactate. The signal stream for the first analyte is then adjusted based on the signal stream for the non-enzyme and the signal stream for the second analyte. Adjusting the first analyte signal stream based on the signal stream for the non-enzyme, the second analyte, or a combination thereof results in more accurate measurements of the first analyte and reduced signal noise. Additionally, the adjustment may be a personalized calibration in that the adjustment is based on the non-enzymes in the same user in which the analytes are being monitored.

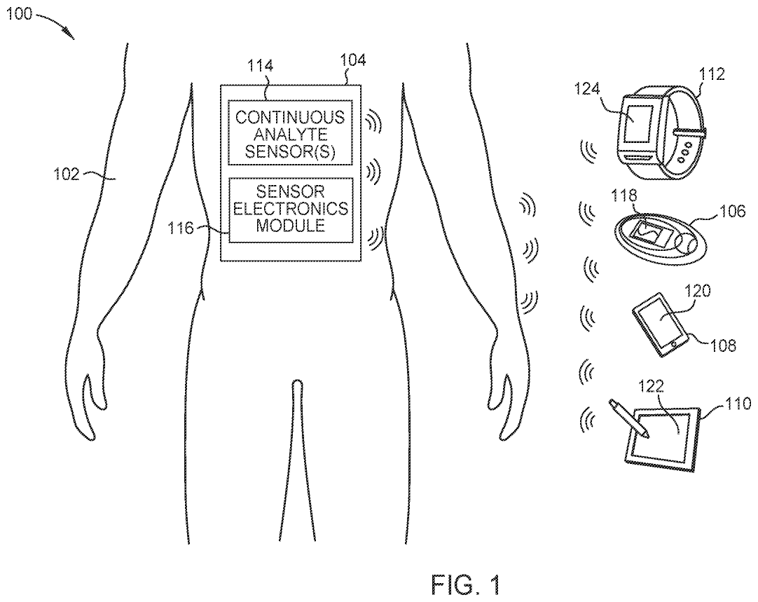

FIG. 1 illustrates an example system 100. As seen in FIG. 1, the system 100 includes a continuous analyte monitoring system 104 (e.g., positioned on a user 102) and display devices 106, 108, 110, and 112. Generally, the continuous analyte monitoring system 104 measures levels of analytes (e.g., substances of interest) in the user 102 and communicates those measured levels to the display devices 106, 108, 110, and 112. In this manner, the continuous analyte monitoring system 104 assists the user 102 with decision support for managing a disease, e.g., diabetes, kidney disease, liver disease, or other types of diseases.

The continuous analyte monitoring system 104 includes one or more continuous analyte sensors 114 (individually referred to herein as continuous analyte sensor 114 and collectively referred to herein as continuous analyte sensors 114) and sensor electronics module 116. The sensor electronics module 116 may be in wired or wireless communication (e.g., directly or indirectly) with one or more of the display devices 106, 108, 110, and 112.

A continuous analyte sensor 114 may include one or more sensors for measuring analytes. A continuous analyte sensor 114 may include a multi-analyte sensor that continuously measures two or more analytes (e.g., glucose, lactate, potassium, ketone, etc.), and/or a single analyte sensor that continuously measures a single analyte. The continuous analyte sensor 114 may be a non-invasive device, a subcutaneous device, a transcutaneous device, a transdermal device, or an intravascular device. The continuous analyte sensor 114 may continuously measure analyte levels of a user using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The continuous analyte sensor 114 may provide a signal stream indicative of the concentration of one or more analytes in the user over time.

An analyte may be a substance or chemical constituent in a biological fluid (for example, blood, interstitial fluid, cerebral spinal fluid, lymph fluid, sweat, or urine) that can be analyzed. Analytes can include naturally occurring substances, artificial substances, metabolites, or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; potassium, quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

The sensor electronics module 116 includes electronic circuitry for measuring, processing, and adjusting signal streams from the continuous analyte sensors 114 (which may be referred to as sensor data). The sensor electronics module 116 can be physically connected to the continuous analyte sensors 114 and can be integral with (non-releasably attached to) or releasably attachable to the continuous analyte sensors 114. The sensor electronics module 116 may include hardware, firmware, or software that enable measurement of levels of analytes via the continuous analyte sensors 114. For example, the sensor electronics module 116 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor.

The display devices 106, 108, 110, and 112 may display sensor data, including measured levels of analytes or adjusted levels of analytes, which may be transmitted by the sensor electronics module 116. The sensor electronics module 116 may transmit raw sensor data that is converted to displayable sensor data via one or more of the display devices 106, 108, 110, and 112. The sensor electronics module 116 may convert raw sensor data to displayable sensor data and transmit the displayable sensor data to one or more of the display devices 106, 108, 110, and 112. Each of the display devices 106, 108, 110, and 112 may include a display such as a touchscreen display 118, 120, 122, or 124 for displaying sensor data to a user or for receiving inputs from the user. For example, a graphical user interface (GUI) may be presented to the user for such purposes. The display devices 106, 108, 110, and 112 may include other types of user interfaces such as a voice user interface instead of, or in addition to, a touchscreen display for communicating sensor data to the user of the display device or for receiving user inputs. The display devices 106, 108, 110, and 112 may display or otherwise communicate the sensor data as it is communicated from the sensor electronics module 116 (e.g., in a customized data package that is transmitted to the display devices 106, 108, 110, and 112 based on their respective preferences).

The display device 106 may include a custom display device specially designed for displaying certain types of displayable sensor data for analyte data received from the sensor electronics module 116. The display device 108 may be a smartphone or a mobile phone using a commercially available operating system (OS) and may display a graphical representation of the continuous sensor data (e.g., including current and historic data). The display device 110 may include a tablet, and the display device 112 may include a smart watch. The display devices 106, 108, 110, and 112 may include a desktop or laptop computer (not shown).

Because different display devices provide different user interfaces, content of the data packages (e.g., amount, format, or type of data to be displayed, alarms, and the like) can be customized (e.g., programmed differently by the manufacture or by an end user) for each particular display device. Accordingly, different display devices can be in direct wireless communication with the sensor electronics module 116 (e.g., such as an on-skin sensor electronics module 116 that is physically connected to continuous analyte sensors 114) during a sensor session to enable a plurality of different types or levels of display or functionality for the displayable sensor information.

One or more of the continuous analyte monitoring system 104 or the display devices 106, 108, 110, and 112 may communicate together wirelessly using one of a variety of wireless communication technologies (e.g., Wi-Fi, Bluetooth, Near Field Communication (NFC), cellular, etc.). A wireless access point (WAP) may be used to couple one or more of the continuous analyte monitoring system 104 or the display devices 106, 108, 110, and 112 to one another. For example, the WAP may provide Wi-Fi, Bluetooth, or cellular connectivity among these devices. NFC may also be used among the devices.

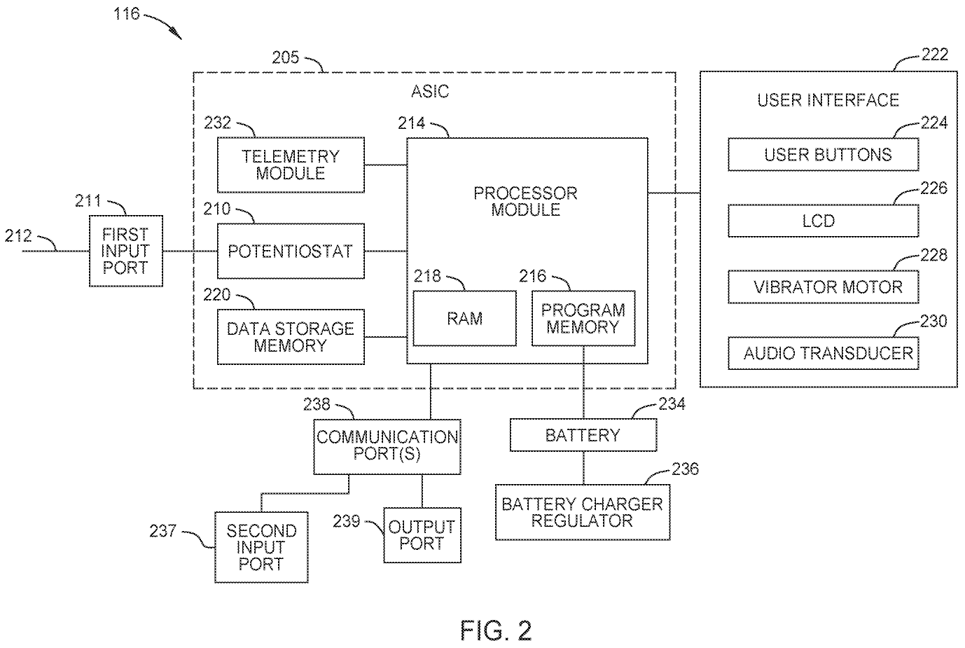

FIG. 2 depicts an example of the sensor electronics module 116, in accordance with some example implementations. The sensor electronics module 116 may include sensor electronics that process sensor information, such as sensor data (e.g., one or more signal streams from analyte sensors of the continuous analyte monitoring system 104), and generate transformed sensor data and displayable sensor information, e.g., via a processor module 214. For example, the processor module 214 may transform sensor data into one or more of the following: filtered sensor data (e.g., one or more filtered analyte concentration values), raw sensor data, calibrated sensor data (e.g., one or more calibrated analyte concentration values), rate of change information, trend information, rate of acceleration/deceleration information, sensor diagnostic information, location information, alarm/alert information, calibration information such as may be determined by factory calibration algorithms as disclosed herein, smoothing or filtering algorithms of sensor data, or the like.

The processor module 214 performs a substantial portion, if not all, of the data processing. The processor module 214 may include a hardware processor or processor circuitry. The processor module 214 may be integral to the sensor electronics module 204 or may be located remotely. The processor module 214 may include smaller subcomponents or submodules. For example, the processor module 214 may include an alert module (not shown) or prediction module (not shown), or any other suitable module that may be utilized to efficiently process data. When the processor module 214 includes submodules, the submodules may be located within the processor module 214, including within the sensor electronics module 204 or other associated devices. For example, the processor module 214 may be located at least partially within a cloud-based analyte processor.

The sensor electronics module 116 may include an application-specific integrated circuit (ASIC) 205 coupled to a user interface 222. The ASIC 205 may be coupled to the user interface 222 through a wireless connection, e.g., Bluetooth or NFC. The ASIC 205 may further include a potentiostat 210, a telemetry module 232 for transmitting data from the sensor electronics module 116 to one or more devices for signal processing and data storage (e.g., processor module 214 and data storage memory 220). Although FIG. 2 depicts ASIC 205, other types of circuitry may be used as well, including field programmable gate arrays (FPGA), one or more microprocessors that provide some (if not all) of the processing performed by the sensor electronics module 204, analog circuitry, digital circuitry, or a combination thereof.

In the example of FIG. 2, through a first input port 211 for sensor data the potentiostat 210 is coupled to a continuous analyte sensor 202, such as a glucose sensor, to generate signal streams indicating levels of the analyte. The potentiostat 210 may also provide via data line 212 a voltage to the continuous analyte sensor 114 to bias the sensor for measurement of a value (e.g., a current and the like) indicative of the analyte concentration in a host (also referred to as the analog portion of the sensor). The potentiostat 210 may have one or more channels depending on the number of working electrodes at the continuous analyte sensor 202.

The potentiostat 210 may include a resistor that translates a current value from the continuous analyte sensor 114 into a voltage value, or a current-to-frequency converter (not shown), which may also continuously integrate a measured current value from the continuous analyte sensor 114 using, for example, a charge-counting device. An analog-to-digital converter (not shown) may digitize the analog signal from the continuous analyte sensor 114 into so-called “counts” to allow processing by the processor module 214. The resulting counts may be directly related to the current measured by the potentiostat 210, which may be directly related to an analyte level, such as a glucose level, in the host.

The telemetry module 232 may be operably connected to the processor module 214 and may provide the hardware, firmware, or software that enable wireless communication between the sensor electronics module 116 and one or more other devices, such as display devices, processors, network access devices, and the like. A variety of wireless radio technologies that can be implemented in the telemetry module 232 include Bluetooth, Bluetooth Low-Energy, ANT, ANT+, ZigBee, IEEE 802.11, IEEE 802.16, cellular radio access technologies, radio frequency (RF), infrared (IR), paging network communication, magnetic induction, satellite data communication, spread spectrum communication, frequency hopping communication, near field communications, or the like. The telemetry module 232 may include a Bluetooth chip, although Bluetooth technology may also be implemented in a combination of the telemetry module 232 and the processor module 214.

The processor module 214 may control the processing performed by the sensor electronics module 204. For example, the processor module 214 may process data (e.g., counts), from the sensor, filter the data, calibrate the data, perform fail-safe checking, or the like.

The processor module 214 may include a digital filter, such as an infinite impulse response (IIR) or a finite impulse response (FIR) filter. This digital filter may smooth a raw data stream received from the continuous analyte sensor 114. Generally, the digital filters are programmed to filter data sampled at a predetermined time interval (also referred to as a sample rate). When the potentiostat 210 measures the analyte (e.g., glucose or the like) at discrete time intervals, these time intervals determine the sampling rate of the digital filter. The potentiostat 210 may measure continuously the analyte, for example, using a current-to-frequency converter. In these current-to-frequency converter implementations, the processor module 214 may be programmed to request, at predetermined time intervals (acquisition time), digital values from the integrator of the current-to-frequency converter. These digital values obtained by the processor module 214 from the integrator may be averaged over the acquisition time due to the continuity of the current measurement. As such, the acquisition time may be determined by the sampling rate of the digital filter.

The processor module 214 may further include a data generator (not shown) that generates data packages for transmission to devices, such as the display devices 106, 108, 110, and 112. Furthermore, the processor module 214 may generate data packets for transmission to these outside sources via the telemetry module 232. In some example implementations, the data packages may, as noted, be customizable for each display device, or may include any available data, such as a time stamp, displayable sensor information, transformed sensor data, an identifier code for the sensor or sensor electronics module 116, raw data, filtered data, calibrated data, rate of change information, trend information, error detection or correction, or the like.

The processor module 214 may also include a program memory 216 and other memory 218. The processor module 214 may be coupled to a communications interface, such as a communication port 238, and a source of power, such as a battery 234. Moreover, the battery 234 may be further coupled to a battery charger or regulator 236 to provide power to sensor electronics module 204 or charge the battery 234.

The program memory 216 may be implemented as a semi-static memory for storing data, such as an identifier for a coupled continuous analyte sensor 114 (e.g., a sensor identifier (ID)) and for storing code (also referred to as program code) to configure the ASIC 205 to perform one or more of the operations/functions described herein. For example, the program code may configure processor module 214 to process signal streams and to adjust a signal stream for one analyte based on a signal from another analyte.

The memory 218 may also be used to store information. For example, the processor module 214 including memory 218 may be used as the system's cache memory, where temporary storage is provided for recent sensor data received from the sensor. In some example implementations, the memory may include memory storage components, such as read-only memory (ROM), random-access memory (RAM), dynamic-RAM, static-RAM, non-static RAM, easily erasable programmable read only memory (EEPROM), rewritable ROMs, flash memory, and the like.

The data storage memory 220 may be coupled to the processor module 214 and may store a variety of sensor information. In some example implementations, the data storage memory 220 stores one or more days of continuous analyte sensor data. For example, the data storage memory may store 1, 2, 2, 4, 5, 6, 7, 8, 9, 10, 11, 12, 13, 14, 15, or 20 days (or more days) of continuous analyte sensor data received from the continuous analyte sensor 114. The stored sensor information may include one or more of the following: a time stamp, raw sensor data (one or more raw analyte concentration values), calibrated data, filtered data, transformed sensor data, or any other displayable sensor information, calibration information (e.g., reference BG values or prior calibration information such as from factory calibration), sensor diagnostic information, and the like.

The user interface 222 wirelessly coupled to the ASIC 205 may include a variety of interfaces, such as one or more buttons 224, a liquid crystal display (LCD) 226, a vibrator 228, an audio transducer (e.g., speaker) 230, a backlight (not shown), or the like. The components that include the user interface 222 may provide controls to interact with the user (e.g., the host). One or more buttons 224 may allow, for example, toggle, menu selection, option selection, status selection, yes/no response to on-screen questions, a “turn off” function (e.g., for an alarm), an “acknowledged” function (e.g., for an alarm), a reset, or the like. The LCD 226 may provide the user with, for example, visual data output. The audio transducer 230 (e.g., speaker) may provide audible signals in response to triggering of certain alerts, such as present or predicted hyperglycemic and hypoglycemic conditions. Audible signals may be differentiated by tone, volume, duty cycle, pattern, duration, or the like. The audible signal may be silenced (e.g., acknowledged or turned off) by pressing one or more buttons 224 on the sensor electronics module 116 or by signaling the sensor electronics module 116 using a button or selection on a display device (e.g., key fob, cell phone, or the like).

Although audio and vibratory alarms are described with respect to FIG. 2, other alarming mechanisms may be used as well. For example, in some example implementations, a tactile alarm is provided including a poking mechanism that “pokes” or physically contacts the patient in response to one or more alarm conditions.

The battery 234 may be operatively connected to the processor module 214 (and possibly other components of the sensor electronics module 116) and provide the necessary power for the sensor electronics module 116. The battery 234 may be a lithium manganese dioxide battery, however any appropriately sized and powered battery can be used (e.g., AAA, nickel-cadmium, zinc-carbon, alkaline, lithium, nickel-metal hydride, lithium-ion, zinc-air, zinc-mercury oxide, silver-zinc, or hermetically-sealed). The battery 234 may be rechargeable. Multiple batteries 234 can be used to power the system. In yet other implementations, the receiver can be transcutaneously powered via an inductive coupling, for example.

A battery charger or regulator 236 may receive energy from an internal or external charger. The battery regulator (or balancer) 236 regulates the recharging process by bleeding off excess charge current to allow all cells or batteries in the sensor electronics module 116 to be fully charged without overcharging other cells or batteries. The battery 234 (or batteries) may be charged via an inductive or wireless charging pad, although any other charging or power mechanism may be used as well.

One or more communication ports 238, also referred to as external connectors, may be provided to allow communication with other devices, for example a PC communication (com) port can be provided to enable communication with systems that are separate from, or integral with, the sensor electronics module 116. The communication port, for example, may include a serial (e.g., universal serial bus or “USB”) communication port, and allow for communicating with another computer system (e.g., PC, personal digital assistant or “PDA,” server, or the like). The sensor electronics module 116 may transmit historical data to a PC or other computing device (e.g., an analyte processor as disclosed herein) for retrospective analysis by a patient or physician. As another example of data transmission, factory information may also be sent to the algorithm from the sensor or from a cloud data source.

The one or more communication ports 238 may further include a second input port 237 and an output port 239 which may be employed to transmit calibrated data, or data to be calibrated, to a display device (e.g., display device 112, 106, 108, 110, etc.). FIG. 2 illustrates these aspects schematically. It will be understood that the ports may be separated physically, but in alternative implementations a single communication port may provide the functions of both the second input port and the output port.

Although separate data storage and program memories are shown in FIG. 2, a variety of configurations may be used as well. For example, one or more memories may be used to provide storage space to support data processing and storage requirements at sensor electronics module 116.

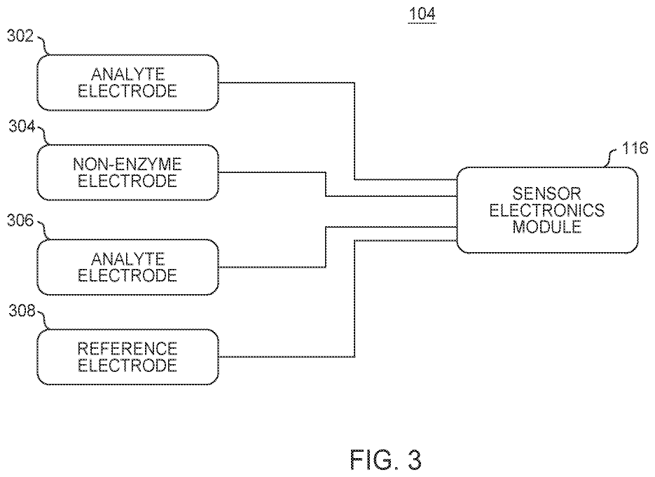

FIG. 3 illustrates an example continuous analyte monitoring systems 104, according to some embodiments disclosed herein. The continuous analyte monitoring system 104 includes a sensor electronics module 116 as well as multiple working electrodes that operate as analyte sensors. As seen in FIG. 3, the continuous analyte monitoring system 104 includes an analyte electrode 302, a non-enzyme electrode 304, an analyte electrode 306, and a reference electrode 308. Each of the electrodes may include a membrane over its surface. The electrodes may be at least partially covered by the same membrane. Alternatively one or more of the electrodes may not be covered by a membrane as in a bare electrode. The electrodes may be electrically coupled to the sensor electronics module 116 (e.g., the potentiostat and the processor module of the sensor electronics module 116).

The electrodes may be formed of any suitable materials and by any suitable methods. For example, the electrodes may be formed of one or more noble metals, such as platinum or platinum/iridium. In some embodiments, the electrodes are carbon-based, and include carbon or carbon/ruthenium. In certain embodiments, the electrodes are formed of graphite, gold, conductive polymer, or the like. The reference electrode 308 may include silver or silver/silver chloride and may be kept currentless. Generally, suitable methods for forming the electrodes include microfabrication techniques, such as physical vapor deposition, chemical vapor deposition, electrodeposition, lithography, and/or etching techniques. Other methods, including spray deposition or dip-coating, are also contemplated.

The continuous analyte monitoring system 104 may be positioned on a body of a user (e.g., user 102) by inserting some or all of the working electrodes into the body. The working electrodes may be inserted into an adequate insertion site, such as an abdomen or an arm of the user 102, where the working electrodes may be in contact with the blood or interstitial fluid of the user 102.

Different electrodes may detect different analytes. For example, each of the electrodes may include an active surface to facilitate electrochemical sensing of desired analytes. The active surface of each electrode may be voxelated, or partitioned into discrete sections (e.g., cubic sections). Different enzymes (not shown), each specific to a different analyte, may be deposited and immobilized on each active surface of each electrode. For example, both glucose oxidase and lactate or uric acid oxidase (and/or other analyte-specific enzymes) may be deposited on the active surface of one electrode. Different enzymes may be immobilized on each voxel of each electrode. In some embodiments, only one type of enzyme is deposited on each voxel of each electrodes. The enzymes may be immobilized via adsorption, entrapment, cross-linking, covalent bonding, or any other suitable immobilization methods.

Each of electrodes includes enzymes for one specific analyte, while different electrodes may include enzymes for different analytes. In certain embodiments, some of the electrodes include two or more enzymes, such as four or more enzymes, which together enable sensing of a single analyte. For example, where an electrode is configured to sense creatinine, the electrode may include four or more enzymes specific for creatinine. An example of a 1-enzyme sensor electrode may include a lactate-specific electrode, and an example of a 2-enzyme sensor electrode may include a ketone-specific electrode. In the example of FIG. 3, the analyte electrode 302 may be used to measure a level of a first analyte (e.g., glucose). The non-enzyme electrode 304 may be used to measure a level of a non-enzyme (e.g., acetaminophen). The analyte electrode 306 may be used to measure a level of a second analyte (e.g., lactate). The reference electrode 308 may have a known electrical potential that may serve as a reference potential when measuring or determining the electrical potential of the other electrodes.

During sensing, the deposited enzymes are utilized to convert a respective analyte to an intermediary product (e.g., hydrogen peroxide), which is then oxidized at the surface of the electrodes. The resulting current flow, which is measured by the potentiostat of the sensor electronics module 116 or an ammeter in communication with the potentiostat, is proportional to the analyte concentration. Examples of suitable enzymes include glucose oxidase for sensing glucose species, lactate oxidase for sensing lactate species, lactose oxidase for sensing lactose species, glutamate oxidase for glutamate species, and the like. In addition to enzymes, the active surfaces may further include immobilized redox mediators (e.g., relays) (not shown), which are small electroactive molecules for shuttling electrons between the enzymes and the electrodes. In some embodiments, the enzymes are immobilized exclusively over the skive regions of electrodes to minimize or avoid cross-talk between different analytes. In other words, an area of active surface over each of the electrodes may be less than a geometric surface area of the respective working electrode. Maintaining a potential bias at the electrodes may facilitate a near-zero peroxide efflux from the skive region with active consumption of the hydrogen peroxide intermediary.

The continuous analyte monitoring system 104 may determine a level (e.g., a concentration) of a first analyte (e.g., glucose) over time using a first analyte signal stream generated using analyte electrode 302, and the continuous analyte monitoring system 104 may generate a non-enzyme signal stream using the non-enzyme electrode 304. The continuous analyte monitoring system 104 may then adjust the level of the first analyte using the non-enzyme signal stream automatically and without user intervention. The level of the first analyte may be adjusted to account for a noise level caused by the non-enzyme. For example, the level of the first analyte may be adjusted using a correction factor, a difference between the first analyte signal stream and the non-enzyme signal stream, or a sensor break-in model with parameters that are adjusted based on the non-enzyme signal stream. As the non-enzyme within the interstitial fluid may interact with the analyte electrode 302 causing signal noise when generating the first analyte signal stream, having a non-enzyme electrode 304 dedicated to generating the non-enzyme signal stream allows for the signal noise to be compensated in the first analyte level.

In a first example, the continuous analyte monitoring system 104 may automatically adjust the level of the first analyte by a correction factor such as a percentage or multiple based on the magnitude of the non-enzyme signal stream. For example, the level of the first analyte at a point in time may be reduced by 10% if the magnitude of a signal value of the non-enzyme signal stream (e.g., at that point in time) is more than 10% of the level of the first analyte. Alternatively, the correction factor may be a multiple. For example, the level of the first analyte at a point in time may be reduced by a multiple 1.5 times a signal value of the non-enzyme signal stream (e.g., at that point in time) if the signal value of the non-enzyme signal stream is more than 50% of a signal value of a baseline non-enzyme signal stream. The baseline non-enzyme signal stream may indicate acceptable non-enzyme levels or interactions with the multi-analyte sensor.

In a second example, the continuous analyte monitoring system 104 may automatically adjust the level of the first analyte by subtracting a signal value of the non-enzyme signal stream from the level of the first analyte or by subtracting a value derived from the signal value of the non-enzyme signal stream from the level of the first analyte. In this manner, a contribution of the noise caused by the non-enzyme may be subtracted or removed from the level of the first analyte.

In a third example, the continuous analyte monitoring system 104 may automatically adjust the level of the first analyte using a sensor break-in model. The sensor break-in model is typically used to adjust the level of the first analyte during a “break-in” period of a sensor, which normally occurs upon initial in vivo use of the sensor (e.g., insertion of the sensor into a user's body). During the break-in period, the signal streams generated by the electrodes of the sensor may be inaccurate and may require compensation to provide accurate measurements of the level of the first analyte. The continuous analyte monitoring system 104 may adjust parameters of the sensor break-in model based on the non-enzyme signal stream. The non-enzyme signal stream may be used to adjust the calibration parameters (e.g., increasing or decreasing the values of the parameter constants of the sensor break-in model that were determined prior to insertion of the continuous analyte monitoring system 104). For example, the calibration parameters may be increased by 10% if the non-enzyme signal stream is 10% above an acceptable threshold. With the adjusted calibration parameters, the sensor break-in model adjusts a generated break-in compensation used during the break-in period of the sensors. The break-in compensation is then used to adjust the level of the first analyte during the break-in period.

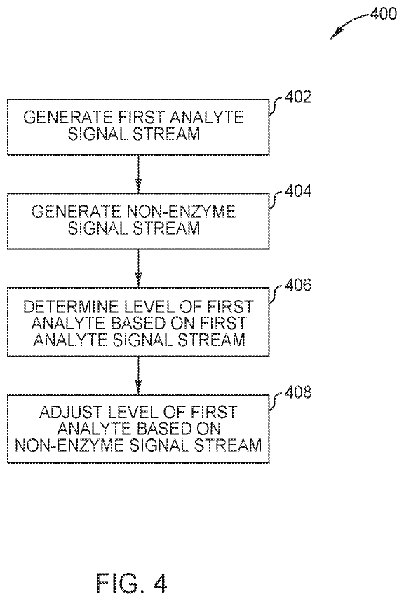

FIG. 4 is a flowchart of an example method 400 for adjusting a determined level of an analyte. In some embodiments, the continuous analyte monitoring system 104 performs the method 400. By performing the method 400, the continuous analyte monitoring system 104 personalizes analyte measurements by adjusting a level of a first analyte of the user based on a non-enzyme signal stream of the user.

At block 402, the continuous analyte monitoring system 104 generates a first analyte signal stream. For example, the continuous analyte monitoring system 104 may use the analyte electrode 302 to continuously or non-continuously monitor a first analyte of a user (e.g., glucose) during a time period. The analyte electrode 302 may be inserted or applied to the user's body and produce an electric current corresponding to the level of the first analyte in the user's body. The electric current may form a signal that changes over time, which forms the first analyte signal stream. The level of the electric current may indicate to the continuous analyte monitoring system 104 a level (e.g., concentration) of the first analyte.

At block 404, the continuous analyte monitoring system 104 continuously or non-continuously generates a non-enzyme signal stream of the user during the time period. For example, the continuous analyte monitoring system 104 may use the non-enzyme electrode 304 to generate the non-enzyme signal stream. The non-enzyme may be, for example, electrochemicals such as acetaminophen. The non-enzyme electrode 304 may be inserted or applied to the user's body and produce an electric current depending on the level of the non-enzyme in the user's body. The electric current may form a signal that changes over time, which forms the non-enzyme signal stream. The level of the electric current may indicate to the multi-analyte sensor 104 a level of the non-enzyme.

At block 406, the continuous analyte monitoring system 104 determines a level of the first analyte based on the first analyte signal stream obtained in block 402. For example, the continuous analyte monitoring system 104 may determine a level of the current produced by the analyte electrode 302. The continuous analyte monitoring system 104 may then translate that current to a level (e.g., concentration) of the first analyte. Thus, the continuous analyte monitoring system 104 may determine a signal value of the first analyte signal stream (e.g., level of the current produced by the analyte electrode 302) at a point in time. The continuous analyte monitoring system 104 may then determine the level of the first analyte measurement (e.g., a concentration of the first analyte) from the signal value. For example, the higher the signal value, the greater level of the first analyte may be.

At block 408, the continuous analyte monitoring system 104 adjusts the level of the first analyte based on the non-enzyme signal stream. The adjustment may involve applying a correction factor to the level of the first analyte, subtracting a signal value of the non-enzyme signal stream or a value derived from the non-enzyme signal stream from the level of the first analyte, or using a sensor break-in model adjusted based on the non-enzyme signal stream. After adjusting the level of the first analyte, the continuous analyte monitoring system 104 may communicate the adjusted level of the first analyte or a version of the adjusted level of the first analyte to one or more of the display devices 106, 108, 110, and 112 for presentation to the user. The continuous analyte monitoring system 104 may then restart the method 400 at block 402.

In applying a correction factor, the continuous analyte monitoring system 104 may adjust the level of the first analyte by a factor such as a percentage or multiple based on the non-enzyme signal stream. For example, the level of the first analyte may be reduced by 10% if a signal value of the non-enzyme signal stream at a point in time is more than 10% of the level of the first analyte. Alternatively, the correction factor may be a multiple. For example, the level of the first analyte may be reduced by a multiple 1.5 times a signal value of the non-enzyme signal stream if the signal value of the non-enzyme signal stream is more than 40% of a signal value of a baseline non-enzyme signal stream. The baseline non-enzyme signal stream may be indicative of acceptable non-enzyme levels or interactions with the multi-analyte sensor.

As another example, the continuous analyte monitoring system 104 may subtract a signal value of the non-enzyme signal stream (e.g., at a point in time) from the level of the first analyte (e.g., at the same point in time). As another example, the continuous analyte monitoring system 104 may subtract a value derived from the non-enzyme signal stream from the level of the first analyte. In this manner, the continuous analyte monitoring system 104 adjusts the level of the first analyte to compensate for the noise caused by the non-enzyme, as indicated by the non-enzyme signal stream.

As another example, the continuous analyte monitoring system 104 may adjust the parameters of a sensor break-in model based on the non-enzyme signal stream. The non-enzyme signal stream may be used to adjust the calibration parameters (e.g., increasing or decreasing the values of the parameter constants) of the sensor break-in model that were determined prior to insertion of the continuous analyte monitoring system 104. For example, the calibration parameters may be increased by 10% if the non-enzyme signal stream is 10% above an acceptable threshold. With the adjusted calibration parameters, the sensor break-in model adjusts a generated break-in compensation used during the break-in period of the sensors. The continuous analyte monitoring system 104 may adjust the level of the first analyte using the break-in compensation during the break-in period.

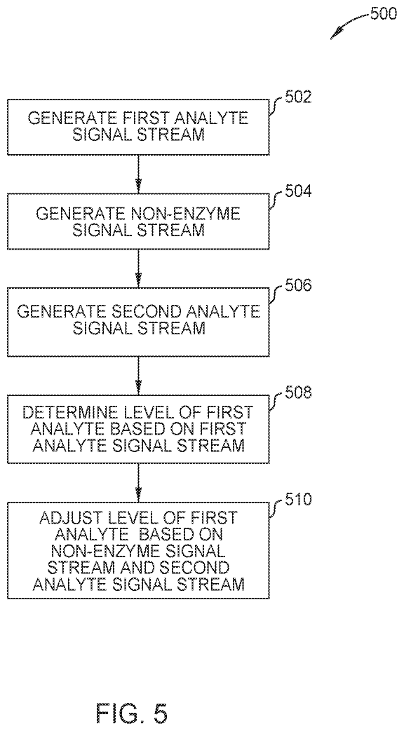

FIG. 5 is a flowchart of an example method 500 for adjusting a determined level of an analyte. In certain embodiments, the continuous analyte monitoring system 104 performs the method 500. By performing the method 500, the continuous analyte monitoring system 104 personalizes analyte measurements by adjusting a level of a first analyte using a non-enzyme signal stream and a second analyte signal stream.

At block 502, the continuous analyte monitoring system 104 generates a first analyte signal stream. The continuous analyte monitoring system 104 may use the analyte electrode 302 to continuously or non-continuously monitor a first analyte of a user (e.g., glucose) during a time period to generate the first analyte signal stream. At block 504, the continuous analyte monitoring system 104 generates a non-enzyme signal stream. The continuous analyte monitoring system 104 may use the non-enzyme electrode 302 to continuously or non-continuously monitor a non-enzyme of the user during the time period to generate the non-enzyme signal stream. At block 506, the continuous analyte monitoring system 104 generates a second analyte signal stream. The continuous analyte monitoring system 104 may use the analyte electrode 306 to continuously or non-continuously monitor a second analyte of the user (e.g., lactate) during the time period to generate the second analyte signal stream.

At block 508, the continuous analyte monitoring system 104 determines the level of the first analyte based on the first analyte signal stream obtained in block 502. For example, the continuous analyte monitoring system 104 may determine a level of the current produced by the analyte electrode 302. The continuous analyte monitoring system 104 may then translate that current to a level (e.g., concentration) of the first analyte.

At block 510, the continuous analyte monitoring system 104 adjusts the level of the first analyte based on the non-enzyme signal stream and the second analyte signal stream. The adjustment may involve (i) applying a correction factor to the level of the first analyte, (ii) subtracting one or more values derived from the second analyte signal stream and the non-enzyme signal stream from the level of the first analyte, (iii) applying a sensor break-in model, or (iv) applying a weighted sum of the first analyte signal stream, the non-enzyme signal stream, and the second analyte signal stream. After adjusting the level of the first analyte, the continuous analyte monitoring system 104 may communicate the adjusted level of the first analyte or a version of the adjusted level of the first analyte to one or more of the display devices 106, 108, 110, and 112 for presentation to the user. The continuous analyte monitoring system 104 may then restart the method 500 at block 502.

In applying a correction factor, the continuous analyte monitoring system 104 may adjust the level of the first analyte by a factor such as a percentage or multiple based on the non-enzyme signal stream and the second analyte signal stream. For example, the level of the first analyte may be reduced by 15% if a signal value of the non-enzyme signal stream at a point in time is more than 10% of a signal value of the first analyte signal stream (e.g., at that point in time) and a signal value of the second analyte signal stream at a point in time is more than 5% of a signal value of the first analyte signal stream (e.g., at that point in time). Alternatively, the correction factor may be a multiple. For example, the level of the first analyte may be reduced by a multiple 1.5 times a sum of signal values of the non-enzyme signal stream and the second analyte signal stream if the signal value of the non-enzyme signal stream is more than 30% of a signal value of a baseline non-enzyme signal stream and the second analyte signal stream is more than 20% of a baseline second analyte signal stream. The baseline non-enzyme signal stream may be indicative of acceptable non-enzyme levels or interactions with the multi-analyte sensor. The baseline second analyte signal stream may be indicative of a typical level of the second analyte.

As another example, the continuous analyte monitoring system 104 may subtract values derived from the non-enzyme signal stream and the second analyte signal stream from the level of the first analyte. For example, signal values of the non-enzyme signal stream and the second analyte signal stream may be subtracted from the level of the first analyte. As another example, the continuous analyte monitoring system 104 may derive values indicative of noise caused by the non-enzyme using the non-enzyme signal stream and the second analyte signal stream. The continuous analyte monitoring system 104 may then subtract these values from the level of the first analyte.

As another example, the continuous analyte monitoring system 104 may adjust the parameters of the sensor break-in model based on the non-enzyme signal stream and the second analyte signal stream. The non-enzyme signal stream and the second analyte signal stream may be used to adjust the calibration parameters (e.g., increasing or decreasing the values of the parameter constants) of the sensor break-in model that were determined prior to insertion of the continuous analyte monitoring system 104. For example, the calibration parameters may be increased by 15% if the non-enzyme signal stream is 10% above an acceptable threshold and if the second analyte signal stream is 5% above a pre-determined threshold. With the adjusted calibration parameters, the sensor break-in model adjusts a generated break-in compensation used during the break-in period of the sensors. The continuous analyte monitoring system 104 may then use the break-in compensation to adjust the level of the first analyte.

As another example, the continuous analyte monitoring system 104 may multiply the first analyte signal stream by a first analyte signal stream weight, the non-enzyme signal stream by a non-enzyme signal stream weight, and the second analyte signal stream by a second analyte signal stream weight. The non-enzyme signal stream weight and the second analyte signal stream weight may be less than the first analyte signal stream weight. For example, the first analyte signal stream weight may be 0.8, the non-enzyme signal stream weight may be 0.05, and the second analyte signal stream weight may be 0.15. The first analyte signal stream weight may be based on signal values of the first analyte signal stream, the non-enzyme signal stream, and the second analyte signal stream. For example, if the magnitude of the non-enzyme signal stream exceeds an acceptable threshold, the non-enzyme signal stream weight is reduced. The second analyte signal stream weight may be similarly adjusted. The first analyte signal stream may then be determined from the non-enzyme signal stream weight and the second analyte signal stream weight. After multiplying the first analyte signal stream, the non-enzyme signal stream, and the second analyst signal stream by their respective weights, the continuous analyte monitoring system 104 may sum these products. The level of the first analyte may then be determined from this sum.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c, b-b, b-b-b, b-b-c, c-c, and c-c-c or any other ordering of a, b, and c).

The term “continuous,” as used herein, is a broad term, and is used in its ordinary sense, and can mean continuous, semi-continuous, continual, periodic, intermittent, regular, etc.

The terms “continuous analyte sensor,” “continuous multi-analyte sensor,” “continuous glucose sensor,” and “continuous lactate sensor,” as used herein, are broad terms, and are used in their ordinary sense, and refer without limitation to a device that continuously measures a concentration of an analyte or calibrates the device (e.g., by continuously adjusting or determining the sensor's sensitivity and background), for example, at time intervals ranging from fractions of a second up to, e.g., 1, 2, or 5 minutes, or longer.

The terms “sensitivity” or “sensor sensitivity,” as used herein, are broad terms, and are used in their ordinary sense, and refer without limitation to an amount of signal produced by a certain concentration of a measured analyte, or a measured species (e.g., H2O2) associated with a measured analyte (e.g., glucose or lactate). For example, a sensor may have a sensitivity of from about 1 to about 300 picoAmps of current for every 1 mg/dL of glucose analyte.

The term “sensor data,” as used herein, is a broad term, and is used in its ordinary sense, and refers without limitation to any data associated with a sensor, such as a continuous analyte or continuous multi-analyte sensor. Sensor data includes a raw data stream, or simply data stream, of analog or digital signal directly related to a measured analyte from an analyte sensor (or other signal received from another sensor), as well as calibrated or filtered raw data. The terms “sensor data point” and “data point” refer generally to a digital representation of sensor data at a particular time. The terms broadly encompass a plurality of time spaced data points from a sensor, such as a continuous analyte sensor, which comprises individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, or any other data associated with a sensor.

The term “sensor electronics,” as used herein, is a broad term, and is used in its ordinary sense, and refers without limitation to components, e.g., hardware or software, of a device configured to process sensor data.

Although certain embodiments herein are described with reference to management of diabetes, diabetes management is only an example of one application for which the present systems and methods may be utilized. The systems and methods described herein can also be used for managing one or more other diseases or conditions, which may or may not include diabetes. For example, the systems and methods described herein can be utilized for managing kidney disease, liver disease, and other types of diseases or conditions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

What is claimed is:

1. An analyte sensor system, comprising:
a first electrode configured to generate a first analyte signal stream;
a second electrode configured to generate a non-enzyme signal stream indicating a level of a non-enzyme over time;
a sensor electronics module configured to:
determine a level of a first analyte based on the first analyte signal stream; and
adjust the level of the first analyte based on the non-enzyme signal stream.

2. The analyte sensor system of claim 1, wherein the sensor electronics module being configured to adjust the level of the first analyte comprises the sensor electronics module being configured to subtract, from the level of the first analyte, a value based on the non-enzyme signal stream.

3. The analyte sensor system of claim 1, wherein the first electrode and the second electrode are separate from each other.

4. The analyte sensor system of claim 1, wherein the first electrode comprises a first electrode at least partially covered by a membrane and the second electrode comprises a second electrode at least partially covered by the membrane.

5. The analyte sensor system of claim 1, wherein the sensor electronics module being configured to adjust the level of the first analyte comprises the sensor electronics module being configured to adjust parameters of a model using the non-enzyme signal stream.

6. The analyte sensor system of claim 1, further comprising a third electrode configured to generate a second analyte signal stream, wherein adjusting the level of the first analyte is further based on the second analyte signal stream.

7. The analyte sensor system of claim 6, wherein the sensor electronics module being configured to adjust the level of the first analyte comprises the sensor electronics module being configured to determine a weighted sum of the first analyte signal stream, the non-enzyme signal stream, and the second analyte signal stream.

8. An analyte sensor system, comprising:
a first electrode covered by a membrane, the first electrode configured to generate a first analyte signal stream;
a second electrode configured to generate a non-enzyme signal stream indicating a level of non-enzymes over time;
a sensor electronics module, configured to:
determine a level of a first analyte based on the first analyte signal stream; and
adjust the level of the first analyte based on the non-enzyme signal stream.

9. The analyte sensor system of claim 8, wherein the second electrode is covered by a second membrane.

10. The analyte sensor system of claim 8, wherein the sensor electronics module being configured to adjust the level of the first analyte comprises the sensor electronics module being configured to subtract, from the level of the first analyte, a value based on the non-enzyme signal stream.

11. The analyte sensor system of claim 8, wherein the sensor electronics module being configured to adjust the level of the first analyte comprises the sensor electronics module being configured to adjust parameters of a model using the non-enzyme signal stream.

12. The analyte sensor system of claim 8, wherein the sensor electronics module being configured to adjust the level of the first analyte comprises the sensor electronics module being configured to apply a correction factor based on the non-enzyme signal stream to the level of the first analyte.

13. The analyte sensor system of claim 8, further comprising a third electrode covered by a second membrane configured to generate a second analyte signal stream indicating a level of a second analyte over time, wherein adjusting the level of the first analyte is further based on the second analyte signal stream.

14. The analyte sensor system of claim 13, wherein the sensor electronics module being configured to adjust the level of the first analyte comprises the sensor electronics module being configured to determine a weighted sum of the first analyte signal stream, the non-enzyme signal stream, and the second analyte signal stream.

15. A method comprising:
generating a first analyte signal stream using a first electrode;
generating a non-enzyme signal stream using a second electrode, wherein the non-enzyme signal stream indicates a level of non-enzymes over time;
determining, using a sensor electronics module, a level of a first analyte based on the first analyte signal stream; and
adjusting, using the sensor electronics module, the level of the first analyte based on the non-enzyme signal stream.

16. The method of claim 15, wherein adjusting the level of the first analyte comprises subtracting a value based on the non-enzyme signal stream from the level of the first analyte.

17. The method of claim 15, further comprising generating a second analyte signal stream using a third electrode, wherein the second analyte signal stream indicates a level of a second analyte over time, and wherein adjusting the level of the first analyte comprises subtracting a value based on the non-enzyme signal stream and a value based on the second analyte signal stream from the level of the first analyte.

18. The method of claim 15, wherein adjusting the level of the first analyte comprises adjusting parameters of a sensor break-in model using the non-enzyme signal stream.

19. The method of claim 15, wherein adjusting the level of the first analyte comprises applying a correction factor based on the non-enzyme signal stream to the level of the first analyte.

20. The method of claim 15, further comprising generating a second analyte signal stream using a third electrode, wherein the second analyte signal stream indicates a level of a second analyte over time, and wherein adjusting the level of the first analyte comprises determining a weighted sum of the first analyte signal stream, the non-enzyme signal stream, and the second analyte signal stream.
